(19) United States
(12) Patent Application Publication
(10) Pub. No.: US 2013/0144194 A1
(43) Pub. Date: Jun. 6, 2013

(54) METHOD AND APPARATUS FOR MAKING ULTRASONIC IRRADIATION PLAN BASED ON ANATOMICAL FEATURES

(71) Applicants: Min-su Ahn, Seoul (KR); Won-chul Bang, Seongnam-si (KR)

(72) Inventors: Min-su Ahn, Seoul (KR); Won-chul Bang, Seongnam-si (KR)

(73) Assignee: Samsung Electronics Co., Ltd., Suwon-si (KR)

(21) Appl. No.: 13/679,824

(22) Filed: Nov. 16, 2012

(30) Foreign Application Priority Data
Nov. 16, 2011 (KR) 10-2011-0119770

(51) Int. Cl. A61N 7/02

(52) U.S. Cl. CPC A61N 7/02
USPC 601/3; 601/2

(57) ABSTRACT

A method of making an ultrasonic irradiation plan includes receiving image data representing anatomical features of a target object, generating information about at least one portion of the target object that is to be irradiated with ultrasound from the image data representing the anatomical features of the target object, and making an ultrasonic irradiation plan for irradiating the target object with ultrasound by simulating irradiating the target object with ultrasound based on the generated information.

### CROSS-REFERENCE TO RELATED APPLICATIONS

This application claims the benefit of Korean Patent Application No. 10-2011-0119770 filed on Nov. 16, 2011, in the Korean Intellectual Property Office, the disclosure of which is incorporated herein by reference in its entirety for all purposes.

### BACKGROUND

1. Field

This disclosure relates to a method and an apparatus for irradiating high-intensity focused ultrasound (HIFU).

2. Description of Related Art

With the progress of medical science, surgery has developed from invasive surgery to minimally invasive surgery for the local treatment of tumors. However, due to revolutionary improvements in technology, high-intensity focused ultrasound (HIFU), which is a noninvasive surgery method, has been recently developed and put into practice. HIFU is a thermal therapy that converts electrical energy into ultrasonic energy, focuses the ultrasonic energy into an ultrasonic beam, irradiates the ultrasonic beam to a specific tissue of a body, and destroys the specific tissue with thermal energy generated by the ultrasonic beam.

Such ultrasonic treatment is considered harmless to a patient since energy is transferred through a medium, unlike radiation treatment, which releases materials that may cause cancer, or may be dangerous in terms of, for example, gene damage. Also, ultrasonic treatment is spotlighted as a harmless treatment for the human body and an environmentally friendly treatment.

### SUMMARY

In one general aspect, a method of making an ultrasonic irradiation plan includes receiving image data representing anatomical features of a target object; generating information about at least one portion of the target object that is to be irradiated with ultrasound from the image data representing the anatomical features of the target object; and making an ultrasonic irradiation plan for irradiating the target object with ultrasound by simulating irradiating the target object with ultrasound based on the generated information.

The generating of the information may include designating any one or any combination of a first portion of the target object that is to be irradiated with the ultrasound, a second portion of the target object that the ultrasound is to avoid, and a third portion of the target object that has a characteristic of disrupting propagation of the ultrasound.

The making of the ultrasonic irradiation plan may include determining whether the ultrasound collides with the second portion or the third portion while virtually irradiating the first portion with the ultrasound using a virtual transducer; and determining a location of the virtual transducer at which irradiating the target object with the ultrasound is allowed based on a result of the determining of whether the ultrasound collides with the second portion or the third portion.

The method may further include moving the virtual transducer to a plurality of locations; the determining of whether the ultrasound collides with the second portion or the third portion may include determining whether the ultrasound collides with the second portion or the third portion at each of the locations of the virtual transducer while virtually irradiating the first portion with the ultrasound at each of the locations of the virtual transducer using the virtual transducer; and the determining of a location of the virtual transducer at which irradiating the target object with the ultrasound is allowed may include determining a plurality of locations of the virtual transducer at which irradiating the target object with the ultrasound is allowed based on a result of the determining of whether the ultrasound collides with the second portion or the third portion at each of the locations of the virtual transducer.

The at least one portion of the target object to be irradiated with the ultrasound may include a plurality of portions of the target object to be irradiated with the ultrasound; the making of the ultrasonic irradiation plan may further include making the ultrasonic irradiation plan by simulating irradiating each of the plurality of portions of the target object with ultrasound based on the generated information; and the moving, the determining of whether the ultrasound collides with the second portion or the third portion, and the determining of a plurality of locations of the virtual transducer at which irradiating the target object with the ultrasound is allowed may be performed for each of the plurality of portions of the target object.

The making of the irradiation plan may further include selecting one of the plurality of locations of the virtual transducer at which irradiating the target object with the ultrasound is allowed may be performed for each of the plurality of portions of the target object based on any one or any combination of an irradiation intensity of an ultrasonic beam irradiated from the virtual transducer, an irradiation time of the ultrasonic beam, and a cooling time for each channel of a plurality of channels of the virtual transducer; and determining a sequential order in which the plurality of portions of the target object are to be irradiated with the ultrasound.

The determining of whether the ultrasound collides with the second portion or the third portion may include determining whether ultrasonic beams radiated to the first portion from all of a plurality of channels of the virtual transducer at the same time collide with the second portion or the third portion.

The determining of whether the ultrasound collides with the second portion or the third portion may include determining, for each channel of a plurality of channels of the virtual transducer, whether an ultrasonic beam radiated to the first portion from one channel of the plurality of channels at a time collides with the second portion or the third portion.

The determining of whether the ultrasound collides with the second portion or the third portion may include determining, for each channel combination of a plurality of different channel combinations of at least two channels of a plurality of channels of the virtual transducer, whether ultrasonic beams radiated to the first portion from the at least two channels at the same time collide with the second portion or the third portion.

The generating of the information may further include designating either one or both of a first critical amount of heat accumulation that will destroy the first portion and a second critical amount of heat accumulation that will destroy the second portion.

The determining of a location of the virtual transducer at which irradiating the target object with the ultrasound is allowed may include calculating a first amount of heat accumulation in the first portion while virtually irradiating the first portion with the ultrasound using the virtual transducer; calculating a second amount of heat accumulation in the second portion while virtually irradiating the first portion with the ultrasound using the virtual transducer; determining whether the first amount of heat accumulation exceeds the first critical amount of heat accumulation; determining whether the second amount of heat accumulation is less than the second critical amount of heat accumulation; and determining the location of the virtual transducer at which irradiating the target object with the ultrasound is allowed based on the result of the determining of whether the ultrasound collides with the second portion or the third portion, a result of the determining of whether the first amount of heat accumulation exceeds the first critical amount of heat accumulation, and a result of the determining of whether the second amount of heat accumulation is less than the second critical amount of heat accumulation.

The generating of the information may include obtaining a movement pattern and a shape-changing pattern of the at least one portion of the target object from the image data.

The making of the ultrasonic irradiation plan may include predicting a location and a shape of the at least one portion of the target object at a point of time at which the target object is to be irradiated with the ultrasound based on the movement pattern and the shape-changing pattern of the at least one portion of the target object.

In another general aspect, a non-transitory computer-readable storage medium stores a program for controlling a computer to perform the method described above.

In another general aspect, an apparatus for making an ultrasonic irradiation plan includes a receiving unit configured to receive image data representing anatomical features of a target object; an information generating unit configured to generate information about at least one portion of the target object that is to be irradiated with ultrasound from the image data representing the anatomical features of the target object; and a plan making unit configured to make an ultrasonic irradiation plan for irradiating the target object with ultrasound by simulating irradiating the target object with ultrasound based on the generated information.

The information generating unit may be further configured to generate the information by designating any one or any combination of a first portion of the target object that is to be irradiated with the ultrasound, a second portion of the target object that the ultrasound is to avoid, and a third portion of the target object that has a characteristic of disrupting propagation of the ultrasound.

In another general aspect, an ultrasonic irradiation method includes receiving first image data representing anatomical features of a target object captured at a first point of time, an ultrasonic irradiation plan for irradiating the target object with ultrasound made based on the first image data, and second image data representing anatomical features of the target object captured at a second point of time; determining whether the ultrasonic irradiation plan made based on the first image data can be used based on a result of comparing the first image data with the second image data; and irradiating the target object with ultrasound according the ultrasonic irradiation plan made based on the first image data when a result of the determining is that the ultrasonic irradiation plan made based on the first image data can be used.

The method may further include making an ultrasonic irradiation plan for irradiating the target object with ultrasound based on the second image data when the result of the determining is that the ultrasonic irradiation plan made based on the first image data cannot be used.

The method may further include modifying the ultrasonic irradiation plan made based on the first image data based on either one or both of third image data representing anatomical features of the target object captured in real time while the target object is being irradiated with the ultrasound, and an amount of heat accumulation due to the irradiating of the ultrasound in at least one portion of the target object where the ultrasound is being irradiated; and irradiating the target object with ultrasound according to the modified ultrasonic irradiation plan.

The determining may include comparing a movement pattern and a shape-changing pattern of at least one portion of the target object where the ultrasound is to be irradiated obtained from the first image data with a movement pattern and a shape-changing pattern of the at least one portion of the target object where the ultrasound is to be irradiated obtained from the second image data; and determining whether the ultrasonic irradiation plan made based on the first image data can be used based on a result of the comparing.

Other features and aspects will be apparent from the following detailed description, the drawings, and the claims.

### BRIEF DESCRIPTION OF THE DRAWINGS

FIG. 1 is a block diagram illustrating an example of an ultrasonic irradiation planning apparatus.

FIG. 2 is a block diagram illustrating an example of an ultrasonic irradiation plan making apparatus illustrated in FIG. 1.

FIG. 3 is a block diagram illustrating an example of the ultrasonic irradiation plan making apparatus illustrated in FIG. 2.

FIG. 4 is a drawing illustrating an example of a process of examining whether a virtual ultrasonic beam radiated from a virtual transducer in a simulation performed by a virtual irradiation performing unit illustrated in FIG. 3 collides with an obstacle that disrupts propagation of the ultrasonic beam in a target object.

FIG. 5 is a drawing illustrating an example of a process of examining whether a virtual ultrasonic beam radiated from at least one channel of the virtual transducer in the simulation performed by the virtual irradiation performing unit illustrated in FIG. 3 collides with an obstacle that disrupts propagation of the ultrasonic beam in the target object.

FIG. 6 is a block diagram illustrating an example of an ultrasonic irradiation performing apparatus illustrated in FIG. 1;

FIG. 7 is a flowchart illustrating an example of an ultrasonic irradiation planning method performed by the ultrasonic irradiation planning apparatus illustrated in FIG. 1.

FIG. 8 is a flowchart illustrating an example of a method of making an ultrasonic irradiation plan performed by the ultrasonic irradiation plan making apparatus illustrated in FIG.

FIG. 9 is a flowchart illustrating an example of a method of performing ultrasonic irradiation performed by the ultrasonic irradiation performing apparatus illustrated in FIG. 1.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent to one of ordinary skill in the art. Also, descriptions of functions and constructions that are well known to one of ordinary skill in the art may be omitted for increased clarity and conciseness.

Throughout the drawings and the detailed description, the same reference numerals refer to the same elements. The drawings may not be to scale, and the relative size, proportions, and depiction of elements in the drawings may be exaggerated for clarity, illustration, and convenience.

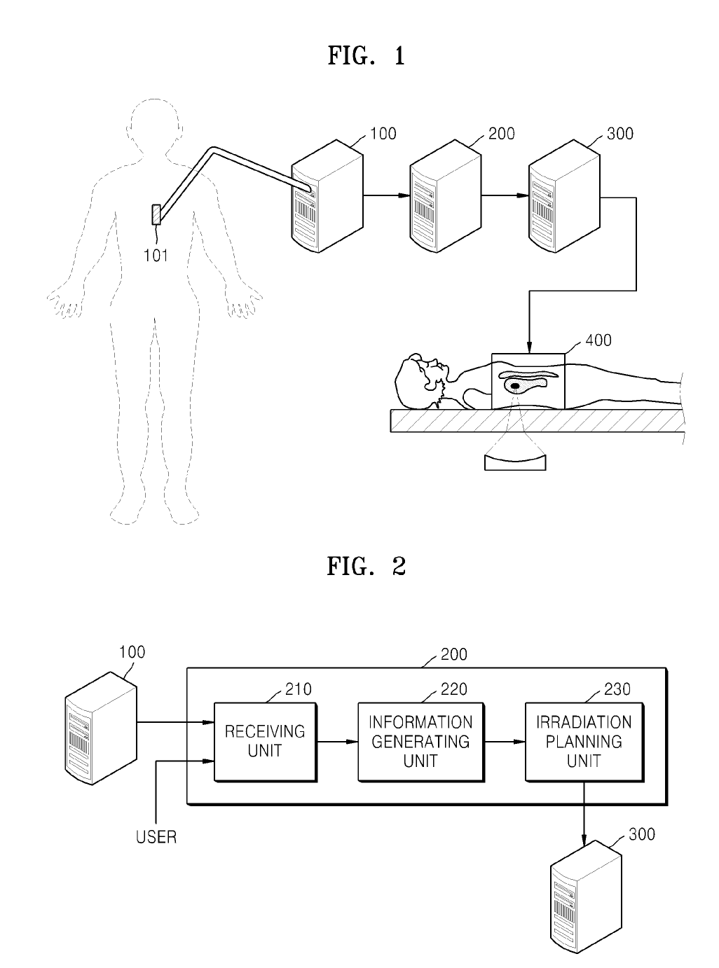

FIG. 1 is a block diagram illustrating an example of an ultrasonic irradiation planning apparatus. Referring to FIG. 1, the ultrasonic irradiation planning apparatus includes a medical imaging instrument 100, an ultrasonic irradiation plan making apparatus 200, an ultrasonic irradiation performing apparatus 300, and an ultrasonic irradiating apparatus 400.

The medical imaging instrument 100 generates medical image data representing anatomical features of a target object by using information about the target object of the ultrasonic irradiation that is detected by a sensor 101 of the medical imaging instrument 100. The anatomical features are structural characteristics inside the target object that may include location, size, and other characteristics of organs in a human body. Examples of the medical imaging instrument 100 include a magnetic resonance imaging (MRI) apparatus, a computed tomography (CT) apparatus, and an ultrasonic imaging apparatus. However, any type of medical imaging instrument known to one of ordinary skill in the art may be used as the medical imaging instrument 100. An image captured by the medical imaging instrument 100 may be a 2-dimensional (2D) image or a 3-dimensional (3D) image.

The ultrasonic irradiation plan making apparatus 200 simulates radiation of ultrasonic energy based on the medical image data representing the anatomical features of the target object, and makes an ultrasonic irradiation plan appropriate for treating the target object based on a result of the simulation. The ultrasonic irradiation performing apparatus 300 determines whether to apply the ultrasonic irradiation plan made by the ultrasonic irradiation plan making apparatus 200 to the target object, and controls the ultrasonic irradiating apparatus 400 in accordance with a result of the determination. The ultrasonic irradiating apparatus 400 generates an ultrasonic beam directly from a transducer and irradiates the ultrasonic beam to the target object in accordance with the ultrasonic irradiation plan made by the ultrasonic irradiation plan making apparatus 200.

FIG. 2 is a block diagram illustrating an example of the ultrasonic irradiation plan making apparatus 200 illustrated in FIG. 1. Referring to FIG. 2, the ultrasonic irradiation plan making apparatus 200 includes a receiving unit 210, an information generating unit 220, and an irradiation planning unit 230.

The receiving unit 210 receives 2D or 3D medical image data representing anatomical features of a target object from the medical imaging instrument 100, and receives data about the target object represented by the medical image data necessary for making the ultrasonic irradiation plan from a user. Examples of the data received from the user may include information about a movement pattern of a lesion, an obstacle, or a normal tissue, information indicating the lesion, the obstacle, or the normal tissue in the target object, information of heat accumulation in the tissue according to the ultrasonic irradiation, and any other information that may be necessary for making the ultrasonic irradiation plan. The information generating unit 220 generates information about at least one portion of the target object where the ultrasound is to be irradiated from the 2D or 3D medical image data representing the anatomical features of the target object that is output from the receiving unit 210.

An ultrasonic irradiation plan has conventionally been directly made by a practitioner according to his or her judgment by directly analyzing a target object using medical image data of the target object. However, it may be difficult for the practitioner to make the ultrasonic irradiation plan for a portion of the target object surrounded by obstacles that block the ultrasound, or a portion of the target object where a movement pattern is complicated. Accordingly, a method of selecting and providing the most appropriate irradiation plan based on an irradiation result after virtually radiating the ultrasound to a complex target object is necessary. Examples that will be described below provide an effective method of automatically making the most appropriate ultrasonic irradiation plan for a target object from the information about the target object generated by the information generating unit 220.

The irradiation planning unit 230 makes at least one ultrasonic irradiation plan based on the information about the target object generated by the information generating unit 220. Specifically, the irradiation planning unit 230 makes the ultrasonic irradiation plan for the target object by performing ultrasonic irradiation on a virtual 3D target object model in a virtual 3D simulation environment conducted on a computer. By performing actual ultrasonic irradiation based on the ultrasonic irradiation plan made using such a method, a lesion may be removed more rapidly and accurately and a normal tissue may be more safely protected compared to when using a conventional method.

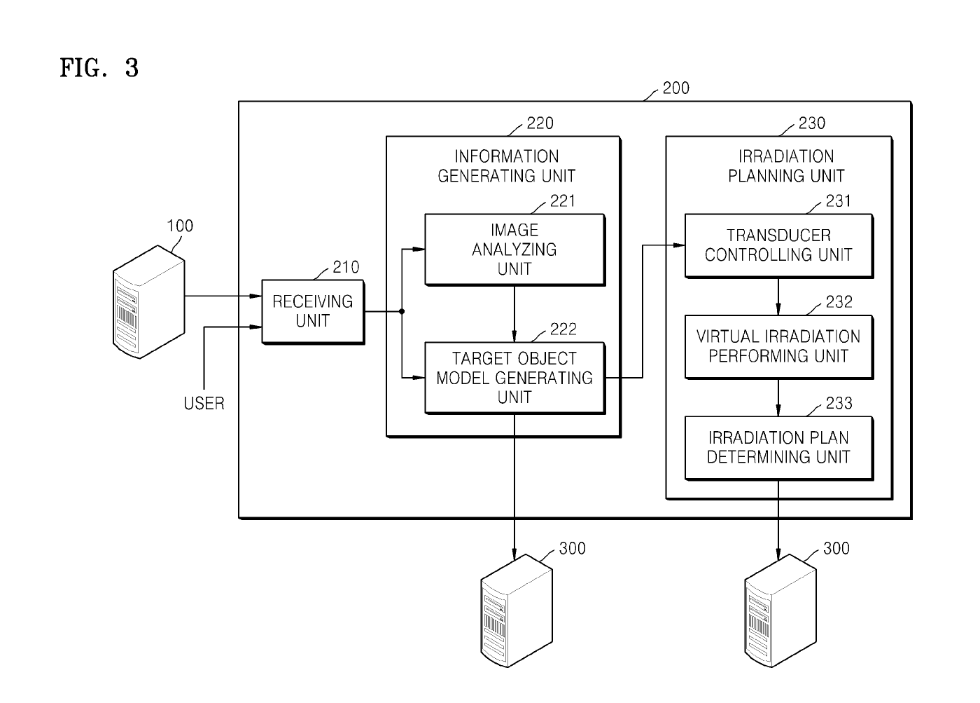

FIG. 3 is a block diagram illustrating an example of the ultrasonic irradiation plan making apparatus 200 illustrated in FIG. 2. Referring to FIG. 3, the information generating unit 220 of the ultrasonic irradiation plan making apparatus 200 includes an image analyzing unit 221 and a target object model generating unit 222, and the irradiation planning unit 230 of the ultrasonic irradiation plan making apparatus 200 includes a transducer controlling unit 231, a virtual irradiation performing unit 232, and an irradiation plan determining unit 233.

In this example, the information generating unit 220 generates a 3D volume of a target object model, which is a target of a virtual ultrasonic irradiation simulation, based on the data received from the user and the medical image data received from the medical imaging instrument 100 via the receiving unit 210.

The image analyzing unit 221 detects the anatomical features of the target object represented by the medical image data by receiving and analyzing the medical image data from the receiving unit 210. The image analyzing unit 221 recognizes a lesion that is a target of the ultrasonic irradiation, an obstacle that disrupts propagation of the ultrasonic beam, and a normal tissue that is to be protected from the ultrasonic irradiation in the target object represented by the medical image data based on the detected anatomical features of the target object.

An example of the lesion may be a malignant tumor, and features of an abnormal tissue of the malignant tumor may be represented by an abnormal shape or an abnormal color in the medical image data. The obstacle is a tissue blocking the ultrasonic beam from reaching the lesion, and when the ultrasonic beam reaches the obstacle, a density of a medium through which the ultrasonic beam is propagating changes rapidly, and thus the ultrasonic beam is reflected or refracted and may not reach the lesion at which it is aimed. An example of the obstacle may be a bone or air. Thus, the ultrasonic beam needs to be radiated along a route that avoids the obstacle. The normal tissue is a tissue that is not to be destroyed by the ultrasonic beam, and may be any tissue other than the lesion. When the ultrasonic beam is radiated to the target object, heat is transferred to nearby tissues as well as a portion where the ultrasonic beam is focused, and accordingly a process of examining whether the normal tissue will not be destroyed by the ultrasonic irradiation is necessary.

For example, the image analyzing unit 221 may detect the anatomical features of the target object represented by the medical image data from a change in brightness of the image, an edge of the image, and other image characteristics of the target object represented by the medical image data. In one example, the image analyzing unit 221 receives the medical image data from the receiving unit 210, generates brightness information of the medical image data, recognizes sections where the brightness information changes rapidly, such as at an edge corresponding to a border of an organ, and identifies the organ by recognizing the location or the shape of the organ based on the border information of the organ. Then, the brightness and shape information generated in the image analyzing unit 221 are compared with brightness and shape information of a normal organ, and when the brightness and shape information generated in the image analyzing unit 221 are deemed to be abnormal due to a difference between the brightness and shape information generated in the image analyzing unit 221 and the brightness and shape information of the normal organ exceeding a fixed margin of error, the organ is be designated as having a lesion. A method of recognizing an organ is not limited to the method described in this example, and any other method known to one of ordinary skill in the art may be used.

Also, the image analyzing unit 221 receives a plurality of the medical image data from the receiving unit 210 and recognizes movement and shape-changing patterns of at least one portion of the organ having the lesion recognized as described above, an obstacle, or a normal tissue from image differences in time between the plurality of the medical image data. In one example, the medical imaging instrument 100 captures multiple frames of the medical image data at time intervals, and the image analyzing unit 221 recognizes organs from the multiple frames of the medical image data, compares how a location and a shape of each of the organs recognized from the multiple frames of the medical image data change over time, and recognizes the movement and shape-changing patterns from the changes.

For example, the image analyzing unit 221 recognizes a movement pattern of a moving organ. An example of a movement of a moving organ may be a movement within a short time period such as a respiratory cycle or a heartbeat, or a movement within a long time period caused by a metastasis or a shape change of a lesion. According to the movement pattern recognized in the image analyzing unit 221, an irradiation location or an irradiation time of the transducer in the ultrasonic irradiation plan generated by the irradiation planning unit 230 may also have a pattern corresponding to the movement pattern.

The target object model generating unit 222 reflects the lesion, obstacle, or normal tissue and the movement and shape-changing patterns thereof recognized by the image analyzing unit 221 in the medical image data received by the receiving unit 210 from the medical imaging instrument 100, and generates a virtual 3D target object model that is a target of the ultrasonic irradiation in a virtual ultrasonic irradiation simulation environment. For example, the target object model generating unit 222 generates a target object model of an image in a 3D volume that represents the target object of the irradiation three-dimensionally, and such a target object model reflects information of the lesion, obstacle, or normal tissue and information of the movement and shape-changing patterns.

In the receiving unit 210, 3D medical image data may be received or 2D medical image data may be received depending on the capability of the medical imaging instrument 100. When 3D medical image data is received in the receiving unit 210, the target object model generating unit 222 designates the lesion, obstacle, or normal tissue recognized by the image analyzing unit 221 in the 3D virtual target object represented by the 3D medical image data received by the receiving unit 210 from the medical imaging instrument 100 and generates the virtual 3D target object model of the designated lesion, obstacle, or normal tissue that moves or changes shape in the 3D virtual target object according to the movement and shape-changing patterns.

When 2D medical image data is received in the receiving unit 210, the target object model generating unit 222 generates 3D medical image data by accumulating a plurality of the 2D medical image data received by the receiving unit 210 from the medical imaging instrument 100, designates the lesion, obstacle, and normal tissue recognized by the image analyzing unit 221 in a 3D virtual target object represented by the 3D medical image data, and generates the virtual 3D target object model of the designated lesion, obstacle, or normal tissue that moves or changes shape in the 3D virtual target object according to the movement and shape-changing patterns.

A critical amount of heat accumulation in a tissue of the target object is an amount of heat accumulation resulting in the tissue of the target object being destroyed by causing the tissue of the target object to stop functioning since more than a certain amount of heat has been accumulated according to the features of the tissue of the target object. Thus, the target object model generating unit 222 receives information about the critical amount of heat accumulation of the organs necessary for generating the virtual 3D target object model from the user through the receiving unit 210 and generates the virtual 3D target object model to reflect such information.

In another example, the target object model generating unit 222 recognizes an organ according to the information received from the user in the receiving unit 210 based on the medical image data received from the medical imaging instrument 100, obtains a movement pattern of the organ according to the received information from the user in the receiving unit 210, and generates a target object model based on the recognized organ and obtained movement pattern.

The transducer controlling unit 231 controls a virtual transducer that virtually radiates a virtual ultrasonic beam in the virtual irradiation performing unit 232 to the virtual 3D target object model generated in the target object model generating unit 222. Such controlling includes controlling a location of the virtual transducer, an irradiation intensity of the virtual ultrasonic beam, an irradiation time of the virtual ultrasonic beam, or any other parameter of the virtual irradiation.

The virtual irradiation performing unit 232 radiates a virtual ultrasonic beam from the virtual transducer that is controlled by the transducer controlling unit 231 to the virtual 3D target object model generated by the target object model generating unit 222, checks whether a lesion is removed or a normal tissue is protected, and determines a location of the transducer where ultrasonic irradiation is allowed based on the a result of the checking whether the lesion is removed or the normal tissue is protected.

A shape of the virtual 3D target object model generated by the target object model generating unit 222 may change according to the movement pattern and the shape-changing pattern of the target object. Accordingly, the virtual irradiation performing unit 232 may predict a shape of the virtual 3D target object model generated by the target object model generating unit 222 at the time of the ultrasonic irradiation, radiate a virtual ultrasonic beam to the virtual 3D target object model having the predicted shape from the virtual transducer that is controlled by the transducer controlling unit 231, check the virtual 3D target object model having the predicted shape to determine whether the lesion is removed or the normal tissue is protected, and determine a location of the transducer where ultrasonic irradiation is allowed based on a result of the checking whether the lesion is removed or the normal tissue is protected.

For example, the virtual irradiation performing unit 232 checks whether the lesion is removed. In this regard, the irradiation performing unit 232 calculates an amount of heat accumulated in each of the organs of the virtual 3D target object model due to the ultrasonic irradiation, compares the calculated amount of accumulated heat with the critical amount of heat accumulation for each of the organs, and then determines the location of the transducer where the ultrasonic irradiation is allowed, the irradiation time, and the irradiation intensity based on a result of comparing the calculated amount of accumulated heat with the critical amount of heat accumulation.

When the amount of heat accumulation is checked for the lesion in the virtual irradiation performing unit 232 illustrated in FIG. 3, the virtual irradiation performing unit 232 calculates the amount of heat accumulated in the lesion due to the ultrasonic beam radiated from the transducer controlled by the transducer controlling unit 231, and determines whether the calculated amount of heat accumulation of the lesion exceeds the critical amount of heat accumulation of the lesion. If the calculated amount of heat accumulation of the lesion exceeds the critical amount of heat accumulation of the lesion, the ultrasonic irradiation is determined to be allowed according to the location of the transducer, the irradiation time, and the irradiation intensity controlled by the transducer controlling unit 231.

When the amount of heat accumulation is checked for the obstacle in the virtual irradiation performing unit 232 illustrated in FIG. 3, the virtual irradiation performing unit 232 checks whether an ultrasonic beam radiated from the transducer controlled by the transducer controlling unit 231 collides with the obstacle, and checks whether the ultrasonic beam reaches the lesion even when the ultrasonic beam collides with the obstacle. If the ultrasonic beam reaches the lesion even when the ultrasonic beam collides with the obstacle, the virtual irradiation performing unit 232 calculates the amount of heat accumulated in the obstacle due to the ultrasonic beam colliding the obstacle, and examines whether the calculated amount of heat accumulation of the obstacle is less than the critical amount of heat accumulation of the obstacle. If the calculated amount of heat accumulation of the obstacle is less than the critical amount of heat accumulation of the obstacle, the ultrasonic irradiation is determined to be allowed according to the location of the transducer, the irradiation time, and the irradiation intensity controlled by the transducer controlling unit 231.

When the amount of heat accumulation is examined in regard to the normal tissue in the virtual irradiation performing unit 232 illustrated in FIG. 3, the virtual irradiation performing unit 232 calculates an amount of heat accumulated in the normal tissue through which the ultrasonic beam passes or the normal tissue near the lesion due to the ultrasonic beam radiated toward the lesion from the transducer controlled by the transducer controlling unit 231, and examines whether the calculated amount of heat accumulation of the normal tissue is less than the critical amount of heat accumulation of the normal tissue. If the calculated amount of heat accumulation of the normal tissue is less than the critical amount of heat accumulation of the normal tissue, the ultrasonic irradiation is determined to be allowed according to the location of the transducer, the irradiation time, and the irradiation intensity controlled by the transducer controlling unit 231.

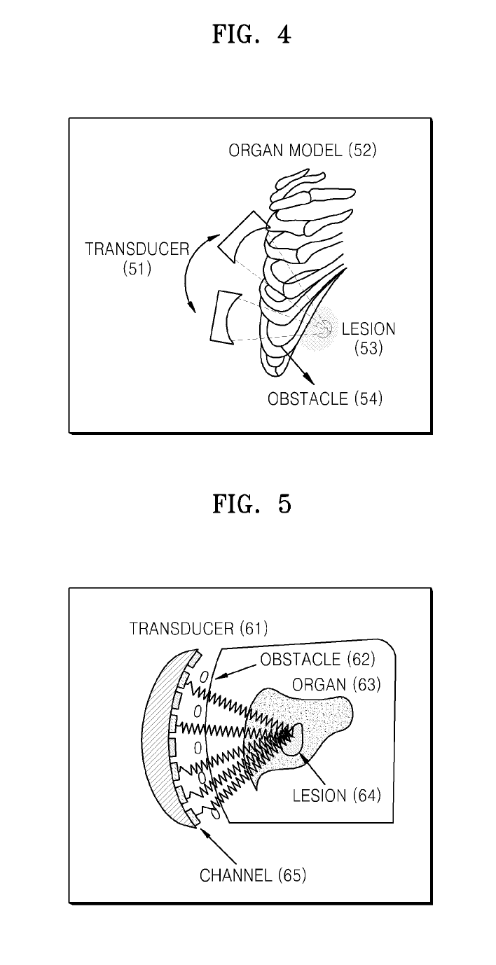

FIG. 4 is a drawing illustrating an example of a process of examining whether a virtual ultrasonic beam radiated from a virtual transducer 51 to an organ model 52 generated by the target object model generating unit 222 illustrated in FIG. 3 in the simulation performed by the virtual irradiation performing unit 232 illustrated in FIG. 3 collides with an obstacle 54 that disrupts propagation of the ultrasonic beam in a target object that is an organ model 52 toward a lesion 53. When the ultrasonic beam collides with the obstacle 54, the location of the virtual transducer 51 is moved.

The simulation in the virtual irradiation performing unit 232 may be repeated many times while sending and receiving information to and from the transducer controlling unit 231. An example of the repeated simulation in the virtual irradiation performing unit 232 is as follows.

For example, the virtual transducer 51 of the simulation performed by the virtual irradiation performing unit 232 is controlled by the transducer controlling unit 231 as the location of the virtual transducer 51 is moved at regular intervals, the virtual irradiation performing unit 232 repeats the simulation according to the movement of the virtual transducer 51 and determines a controlling method of the virtual transducer 51 for which the ultrasonic irradiation is allowed according to a result of the repeated simulation.

In another example, the transducer controlling unit 231 may independently control whether an ultrasonic beam radiated from each of a plurality of channels of the virtual transducer 51 is to be radiated or not from each channel, and thus the virtual transducer 51 may be controlled to radiate the ultrasonic beam from a channel combination of at least one or more channels, and the simulation performed by the virtual irradiation performing unit 232 may be repeated as the transducer controlling unit changes the channel combination.

The transducer controlling unit 231 may determine the channel combination according to various methods. Such a combination may be a combination radiating an ultrasonic beam from one channel, a combination radiating ultrasonic beams from a plurality of channels fewer than all of the channels, or a combination radiating ultrasonic beams from all of the channels. Hereinafter, such methods will be described in detail with respect to FIG. 5.

FIG. 5 is a drawing illustrating a process of examining whether a virtual ultrasonic beam radiated from at least one of channels 65 of a virtual transducer 61 in the simulation performed by the virtual irradiation performing unit 232 collides with an obstacle 62 that disrupts propagation of the ultrasonic beam in the target object toward a lesion 64 in an organ 63. When the ultrasonic beam collides with the obstacle 62, a channel combination of the transducer 61 radiating the ultrasonic beam is changed.

The transducer controlling unit 231 illustrated in FIG. 3 controls the ultrasonic beam to be radiated from some of the plurality of channels 65 of the transducer 61 as described above. Such controlling is to ensure the ultrasonic beam does not collide with the obstacle 62. An example of the obstacle 62 may be a bone.

Accordingly, an example of a simulation performed in the virtual irradiation performing unit 232 as the channel combination of the transducer 61 changes is as follows. The virtual irradiation performing unit 232 checks whether each of the ultrasonic beams radiated while focusing on the lesion 64 from all the channels 65 of the transducer 61 collides with the obstacle 62, and determines a channel combination of the transducer 61 for which the ultrasonic irradiation is allowed based on a result of the checking.

In another example, the virtual irradiation performing unit 232 illustrated in FIG. 3 checks whether the ultrasonic beam radiated to the lesion 64 from one channel out of all the channels 65 of the transducer 61 collides with the obstacle 62. The simulation is repeated as the channel 65 that radiates the ultrasonic beam changes, and a channel combination of the transducer 61 for which the ultrasonic irradiation is allowed is determined based on a result of the checking.

In another example, the virtual irradiation performing unit 232 illustrated in FIG. 3 checks whether each of at least two or more of the ultrasonic beams radiated while focusing on the lesion 64 from all the channels 65 of the transducer 61 collide with the obstacle 62. The simulation is repeated as the channels 65 that radiate the ultrasonic beams change, and a channel combination of the transducer 61 for which the ultrasonic irradiation is allowed is determined based on a result the checking.

When a lesion area is larger than an area where the ultrasonic beam is focused, or when a plurality of lesions are present, treatment cannot be accomplished by destroying the lesions by focusing on them a single time, and thus focusing and performing the ultrasonic irradiation many times will be necessary. Accordingly, the transducer repeats the ultrasonic irradiation until all the lesions are destroyed by changing the location of the focus. The virtual irradiation performing unit 232 also repeats radiating the virtual ultrasonic beam to each of the focuses as the locations of the focuses change when radiating the virtual ultrasonic beam, and ends the repetition when the entire lesion present in the target object is determined as being destroyed. The controlling method of the transducer for which the ultrasonic irradiation is allowed that is determined in the virtual irradiation performing unit 232 according to the virtual ultrasonic irradiation result may be determined to be controlling methods corresponding to each of the focuses. The controlling method is a method of controlling the location of the transducer, the irradiation time of the ultrasonic beam, the irradiation intensity, and the channel combination.

In one example, the irradiation plan determining unit 233 obtains a controlling method of the location of irradiation of the transducer, the irradiation time of the ultrasonic beam, the irradiation intensity, the channel combination from which the ultrasonic beam is radiated that is determined in the virtual irradiation performing unit 232, and based thereon, finally determines an optimized ultrasonic irradiation plan to be provided to the practitioner.

In another example, the irradiation plan determining unit 233 obtains a controlling method of the location of irradiation of the transducer, the irradiation time of the ultrasonic beam, the irradiation intensity, and the channel combination corresponding to each of the focuses where the ultrasonic beam is irradiated that is determined based on the repeated simulation according to a change of focus performed by the virtual irradiation performing unit 232, and based thereon, finally determines an optimized ultrasonic irradiation plan to be provided to the practitioner.

When the ultrasonic irradiation is performed on a plurality of focuses, the irradiation plan determining unit 233 determines an optimized ultrasonic irradiation plan that may be performed on all the focuses. Accordingly, the irradiation plan determining unit 233 determines a controlling method of the location of the ultrasonic irradiation of a virtual transducer, the ultrasonic irradiation intensity, the ultrasonic irradiation time, the channel combination that radiates the ultrasonic beam, and a sequential order of irradiating the plurality of focuses as mentioned above by considering the total time spent in the ultrasonic irradiation or a moving distance of the virtual transducer, and based thereon, finally determines an optimized ultrasonic irradiation plan to be provided to the practitioner.

For example, the irradiation plan determining unit 233 may determine an optimized ultrasonic irradiation plan based on a shortest irradiation time. Alternatively, the irradiation plan determining unit 233 may determine an optimized ultrasonic irradiation plan based on a shortest cooling time. Alternatively, the irradiation plan determining unit 233 may determine an optimized ultrasonic irradiation plan based on a plurality of conditions depending on the circumstances under which the ultrasonic irradiation of the target object is to be performed.

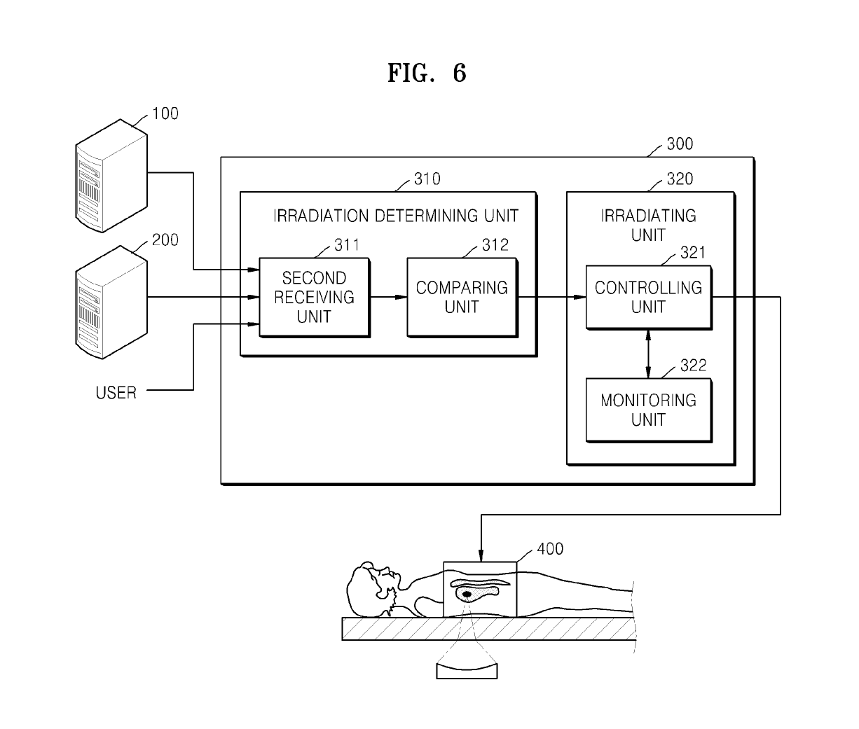

FIG. 6 is a block diagram illustrating an example of the ultrasonic irradiation performing apparatus 300 illustrated in FIG. 1. Referring to FIG. 6, the ultrasonic irradiation performing apparatus 300 includes an irradiation determining unit 310 and an irradiating unit 320. The irradiation determining unit 310 includes a second receiving unit 311 and a comparing unit 312, and the irradiating unit 320 includes a controlling unit 321 and a monitoring unit 322.

The comparing unit 312 receives past information representing anatomical features of the target object at a point of time in the past, the ultrasonic irradiation plan made by the ultrasonic irradiation plan making apparatus 200 based on the past information, and present information representing the anatomical features of the target object at a present point of time when the treatment is to be performed from the second receiving unit 311, compares the present information with the past information, and determines whether to perform the ultrasonic irradiation of the target object at the present point of time according to the ultrasonic irradiation plan made by the ultrasonic irradiation plan making apparatus 200 based on the past information according to a result of the comparison. Examples of the past and present information include the medical image data generated by the medical imaging instrument 100, the target object model generated by the target object model generating unit 222, and any other information represent past and present states of the target object.

In an example in which the past and present information is medical image data generated by the medical imaging instrument 100, the second receiving unit 311 receives past medical image data representing the anatomical features of the target object that was used in the simulation performed by the ultrasonic irradiation plan making apparatus 200 and the ultrasonic irradiation plan made by the ultrasonic irradiation plan making apparatus 200 based on the past medical image data from the ultrasonic irradiation plan making apparatus 200, and receives present medical image data representing the anatomical features of the target object at the present point of time from the medical imaging instrument 100. The comparing unit 312 receives the past medical image data representing the anatomical features of the target object that was used in the simulation performed by the ultrasonic irradiation plan making apparatus 200, the ultrasonic irradiation plan made by the ultrasonic irradiation plan making apparatus 200 based on the past medical image data, and the present medical image data representing the anatomical features of the target object at the present point of time from the receiving unit 311, compares the present medical image data with the past medical image data, and determines whether to perform the ultrasonic irradiation of the target object at the present point of time according to the ultrasonic irradiation plan made based on the past medical image data according to a result of the comparison.

In an example in which the past and present information is a target object model generated by the target object model generating unit 222, the second receiving unit 311 receives a past target object model generated by the target object model generating unit 222 based on past medical image data representing anatomical features of the target object, an ultrasonic irradiation plan made by the ultrasonic irradiation plan making apparatus 200 based on the past target object model, and a present target object model generated by the target object model generating unit 222 based on present medical image data representing the anatomical features of the target object. The comparing unit 312 receives the past target object model generated by the target object model generating unit 222 based on the past medical image data, the ultrasonic irradiation plan made by the ultrasonic irradiation plan making apparatus 200 based the past target object model, and the present target object model generated by the target object model generating unit 222 based on the present medical image data, compares organs, movement thereof, and shape-changing patterns thereof in the past target object model and the present target object model, determines not to perform the ultrasonic irradiation of the target object according to the ultrasonic irradiation plan made based on the past target object model at the present point of time when a difference obtained by the comparison exceeds a fixed margin of error, and ends the process to make a new irradiation plan.

When the difference obtained by the comparison does not exceed the fixed margin of error, the comparing unit 312 determines to perform the ultrasonic irradiation of the target object of the according to the ultrasonic irradiation plan made based on the past target object model at the present point of time, and provides the ultrasonic irradiation plan to the controlling unit 321.

The controlling unit 321 receives the ultrasonic irradiation plan from the comparing unit 312, and controls the ultrasonic irradiating apparatus 400 according to the ultrasonic irradiation plan. In one example, the ultrasonic irradiation plan received from the comparing unit 312 includes information of a location of a focus of an ultrasonic beam radiated from the transducer, a location of the transducer, an irradiation intensity of the ultrasonic beam, an irradiation time, a channel combination generating the ultrasonic beam, and a sequential order of irradiating the ultrasonic beam at a plurality of focuses when radiated from the transducer to the target object, and the controlling unit 321 controls the transducer of the ultrasonic irradiating apparatus 400 according to the irradiation plan.

In one example, the monitoring unit 322 may obtain real-time image data while the ultrasonic irradiation is being performed as the controlling unit 321 controls the ultrasonic irradiating apparatus 400, and may order the controlling unit 321 to change the ultrasonic irradiation plan based on the real-time image data. In another example, the monitoring unit 322 obtains a temperature and an amount of heat accumulation of an organ in the target object in real time while the ultrasonic irradiation is being performed as the controlling unit 321 controls the ultrasonic irradiating apparatus 400, checks whether the entire lesion tissue has been destroyed and whether normal tissue is being protected based on the temperature and the amount of heat accumulation, and may order the controlling unit 321 to change the ultrasonic irradiation plan based on a result of the checking.

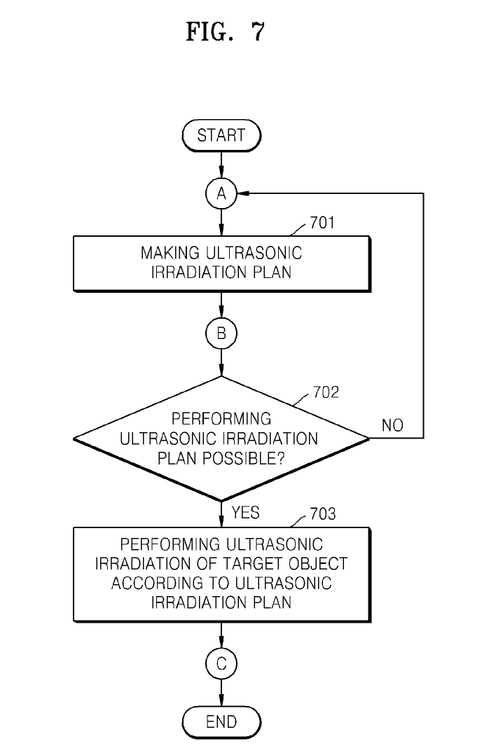

FIG. 7 is a flowchart illustrating an example of an ultrasonic irradiation planning method performed by the ultrasonic irradiation planning apparatus illustrated in FIG. 1. Referring to FIG. 7, the ultrasonic irradiation planning method includes operations performed in sequence by the ultrasonic irradiation planning apparatus 200 illustrated in FIG. 1. Therefore, although the description of the ultrasonic irradiation planning apparatus illustrated in FIG. 1 provided above is omitted below for conciseness, the description is also applicable to the ultrasonic irradiation planning method illustrated in FIG. 7.

In operation 701, an ultrasonic irradiation plan is made. In operation 702, whether to perform the ultrasonic irradiation plan made in 701 is determined. When it is determined in operation 702 not to perform the ultrasonic irradiation plan made in operation 701, the process returns to operation 701 to make a new ultrasonic irradiation plan. When it is determined in operation 702 to perform the ultrasonic irradiation plan made in operation 701, ultrasonic irradiation of the target object is performed according to the ultrasonic irradiation plan made in operation 701 in operation 703.

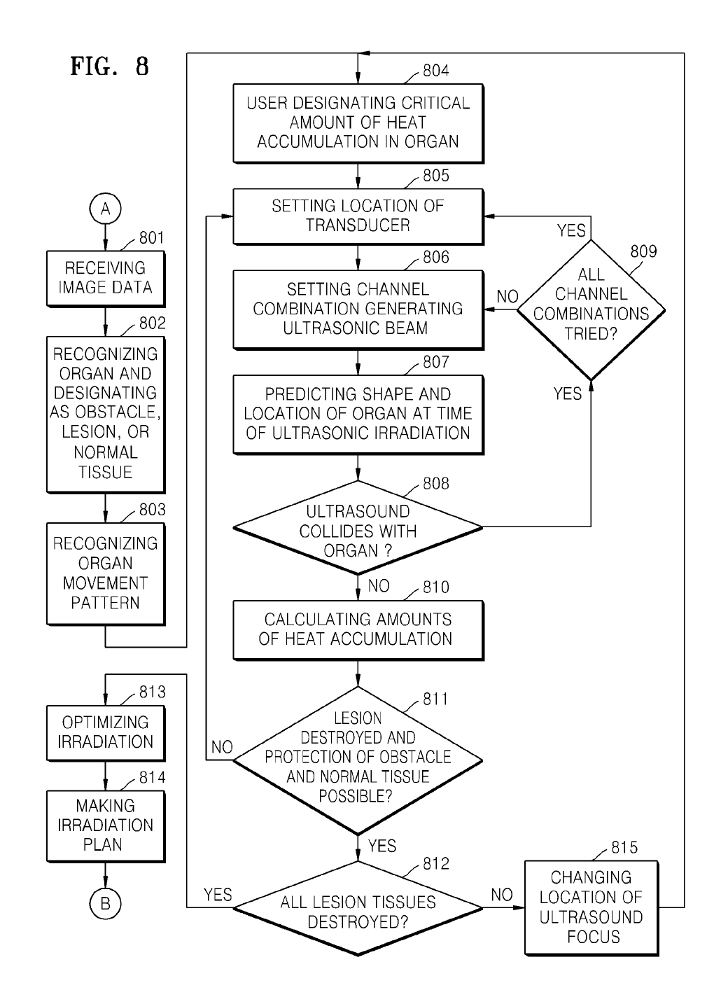

FIG. 8 is a flowchart illustrating an example of a method of making the ultrasonic irradiation plan performed by the ultrasonic irradiation plan making apparatus 200 illustrated in FIG. 1, and is a detailed flowchart of operation 701 illustrated in FIG. 7. Referring to FIG. 8, the method of making the ultrasonic irradiation plan performed by the ultrasonic irradiation plan making apparatus 200 includes the operations described below.

In operation 801, the medical image data generated by the medical imaging instrument 100 is received via the receiving unit 210. In operation 802, an organ is recognized based on the medical image data received in operation 801, and the recognized organ is designated as an obstacle, a lesion, or a normal tissue. Although various operations in FIG. 8 refer to an organ for conciseness of description, there may be a plurality of organs or a plurality of portions of organs that are recognized and designated as an obstacle, a lesion, or a normal tissue. In operation 803, a movement pattern of the organ designated in operation 802 is recognized. In operation 804, a critical amount of heat accumulation of the designated organ is designated. In operation 804, a user may directly input the critical amount of heat accumulation of the organ, or the critical amount of heat accumulation of the organ may be obtained from a storage device of a computer where it is stored. Thus, a target object model to be used in performing simulation of ultrasonic irradiation on a computer is constructed.

In operation 805, in radiating an ultrasonic beam from a virtual transducer to the target object model, a location of the transducer is set. In operation 806, in radiating the ultrasonic beam from each of a plurality of channels of the transducer, from which channel or channels the ultrasonic beam is to be generated is set, that is, a channel combination of at least one or more channels to generate the ultrasonic beam among a plurality of channels is set. Although the description of a method of setting the channel combination in the description of the ultrasonic irradiation plan making apparatus 200 in FIG. 1 provided above is omitted below for conciseness, the description is also applicable to the method of making the ultrasonic irradiation plan illustrated in FIG. 8.

In operation 807, a shape and a location of the organ in the target object model at the time of the ultrasonic irradiation is predicted based on the movement pattern recognized in operation 803, and the predicted information is applied to the target object model that is a target of the ultrasonic irradiation of the virtual transducer.

In operation 808, when a virtual ultrasonic beam is radiated to the target object model predicted in operation 807 according to the channel combination set in operation 806 from the location of the transducer set in operation 805, it is determined whether the virtual ultrasonic beam collides with an organ designated as an obstacle in the target object model. When the ultrasonic beam collides with the organ designated as an obstacle in the target object model, the location and the channel combination of the transducer are changed by repeating operations 805 through 808 under control of operation 809. In operation 809, it is determined if all channel combinations of the transducer have been tried. If it is determined in operation 809 that not all channel combinations of the transducer have been tried, a new channel combination is set in operation 806, and operations 807 and 808 are repeated. If it is determined in operation 809 that all channel combinations of the transducer have been tried, a new location of the transducer is set in operation 805, and operations 806 though 808 are repeated. When the ultrasonic beam does not collide with the organ designated as an obstacle in the target object model, the method of making the ultrasonic irradiation plan proceeds to operation 810.

In operation 810, amounts of heat accumulation due to the virtual ultrasonic beam irradiation of the lesion, obstacle, and normal tissue of the target object model are calculated by performing a simulation. In operation 811, it is determined whether the amount of heat accumulation of the lesion calculated in operation 810 exceeds the designated critical amount of heat accumulation of the lesion of the target object model, thereby determining whether the lesion has been destroyed. Also, it is determined whether the amounts of heat accumulation of the obstacle and the normal tissue calculated in operation 810 are less than the designated critical amounts of heat accumulation of the obstacle and the normal tissue of the target object model, thereby determining whether it is possible to protect the obstacle and the normal tissue. When it is determined in operation 811 that the lesion has not been destroyed, or that is not possible to protect the obstacle and the normal tissue, the location of the transducer and the channel combination of the transducer are changed by repeating operations 805 through 811. When it is determined in operation 811 that the lesion has been destroyed, and that it is possible to protect the obstacle and the normal tissue, the method proceeds to operation 812.

In operation 812, it is determined whether all lesion tissues have been destroyed when it is necessary to perform focusing a plurality of times since a lesion area is larger than an area of one focus formed by the ultrasonic beam radiated from the transducer, or when lesions are present in a plurality of different locations. When it is determined in operation 812 that not all lesion tissues have been destroyed, the method proceeds to operation 815. In operation 815, operations 804 through 812 are repeated by changing the location where the ultrasonic beam is focused, that is a location where the ultrasonic beam reaches. When it is determined in operation 812 that all lesion tissues have been destroyed, the method proceeds to operation 813.

In operation 813, controlling methods of the transducer corresponding to each of the plurality of focus locations are obtained, and the most appropriate controlling method is determined as an optimized controlling method based on the irradiation intensity of the ultrasonic beam, the irradiation time, or a cooling time for decreasing the temperature of the tissues that is increased due to the ultrasonic irradiation of each of the controlling methods.

For example, the operation 813 may determine a controlling method having shortest irradiation time as the optimized controlling method. Alternatively, the operation 813 may determine a controlling method having a shortest cooling time as the optimized controlling method.

In operation 814, based on the most appropriate controlling method determined in operation 813, an order of sequentially radiating the ultrasonic beam to the plurality of the focus locations by moving the transducer is determined, and finally the ultrasonic irradiation plan is made.

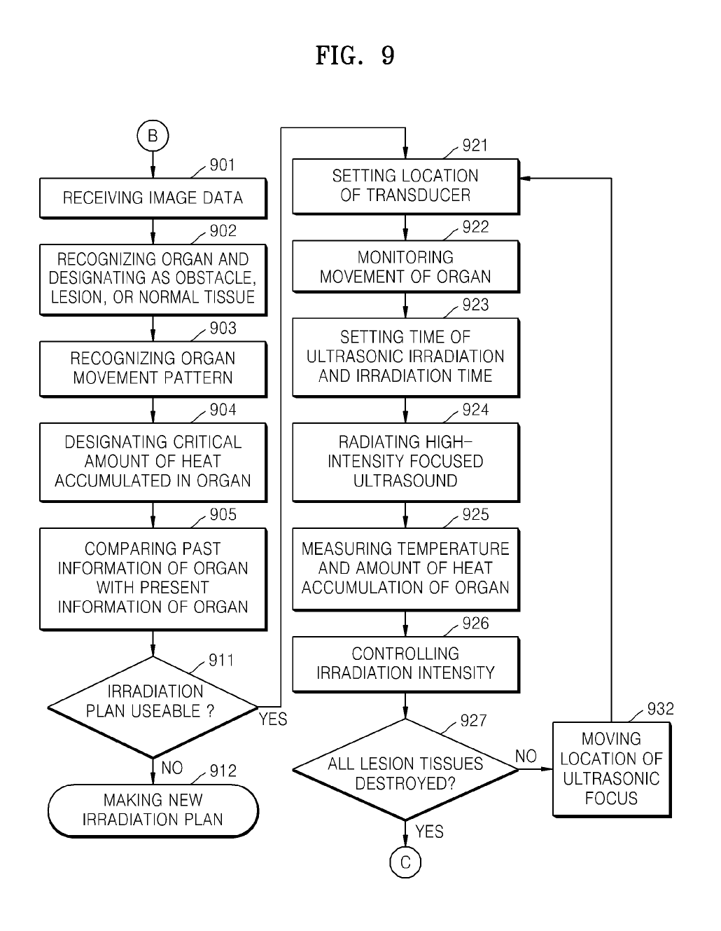

FIG. 9 is a flowchart illustrating an example of a method of performing the ultrasonic irradiation performed by the ultrasonic irradiation performing apparatus 300 illustrated in FIG. 1, and is a detailed flowchart of operations 702 and 703 illustrated in FIG. 7. Referring to FIG. 9, the method of performing the ultrasonic irradiation performed by the ultrasonic irradiation performing apparatus 300 includes the operations described below.

In operation 901, present medical image data generated by the medical imaging instrument 100 is received via the second receiving unit 311 illustrated in FIG. 6. The present medical image data is captured at a time when the ultrasonic irradiation is to be performed, which is later than a time at which the past medical image data used in making the ultrasonic irradiation plan was captured.

In operation 902, an organ is recognized based on the present medical image data received in operation 901, and is designated as a lesion, an obstacle, or a normal tissue. Although various operations in FIG. 9 refer to an organ for conciseness of description, there may be a plurality of organs or a plurality of portions of organs that are recognized and designated as an obstacle, a lesion, or a normal tissue. In operation 903, a movement patterns of the organ designated in operation 902 is recognized. In operation 904, a critical amounts of heat accumulation of the designated organ is designated. In operation 904, a user may directly input the critical amount of heat accumulation of the organ, or the critical amount of heat accumulation may be obtained from a storage device of a computer where it is stored. Thus, a present target object model of the target object at the time when the ultrasonic irradiation is to be performed is constructed.

In operation 905, a past target object model generated based on the past medical image data and the ultrasonic irradiation plan made based on the past target object model are received from the ultrasonic irradiation plan making apparatus 200 via the second receiving unit 311, past information of the organ of the past target object model and a movement pattern thereof are compared with present information of the organ of the present target object model and a movement patterns thereof, and a difference therebetween is calculated.

In operation 911, based on the difference calculated in operation 905, it is determined whether the ultrasonic irradiation plan made based on the past target object model generated based on the past medical image data is applicable to the target object, which is a target of the present medical image data. For example, it is determined whether a difference in a location of the organ in the movement pattern of the organ measured in operation 905 exceeds a fixed margin of error. If the difference exceeds the fixed margin of error, it is determined that the ultrasonic irradiation plan made based on the past target object model cannot be used, and the difference does not exceed the fixed margin of error, it is determined that the ultrasonic irradiation plan made based on the past target object model can be used.

When it is determined in operation 911 that the ultrasonic irradiation plan cannot be used, the method proceeds to operation 912, and the method ends to make a new ultrasonic irradiation plan. When it is determined in operation 911 that the ultrasonic irradiation plan can be used, the method proceeds to operation 921.

In operation 921, the location of the transducer of the ultrasonic irradiating apparatus 400 illustrated in FIGS. 1 and 6 is set according to the ultrasonic irradiation plan, and in operation 922, the movement of the organ of the target object that is the object of the ultrasonic irradiation is monitored in real time. In such a monitoring operation, the ultrasonic irradiation plan may be modified by receiving new image data representing the anatomical features of the target object in real time while the ultrasonic beam is being radiated, recognizing the anatomical features of the target object from the new image data, and modifying the ultrasonic irradiation plan to reflect any changes in the anatomical features of the target object.

Also, according to the information of the ultrasonic irradiation plan received in operation 901, an operation of changing the time of ultrasonic irradiation from the transducer, the irradiation intensity, or the channel combination of the transducer as well as moving the location of the transducer may be included in operation 921.

In operation 923, a point of time at which the ultrasonic irradiation is to be performed and a period of time for which the irradiation is to be performed are set according to a result of the monitoring in operation 922.

In operation 924, according to control of the transducer of the ultrasonic irradiating apparatus 400 that is controlled in operations 921 through 924, high-intensify focused ultrasound (HIFU) is radiated to the target object. In operation 925, the temperature and the amount of heat accumulation of the organ in the target object are measured in real time while the ultrasonic irradiation is being performed in operation 924.

In operation 926, the irradiation intensity of the ultrasonic beam is controlled based on the temperature and the amount of heat accumulation of the organ measured in operation 924 by determining whether the amount of heat accumulated in the lesion tissue where the focus of the ultrasonic irradiation is located exceeds the critical amount of heat accumulated in the lesion tissue determining whether the amount of heat accumulated in each of the obstacle and the normal tissue is less than the critical amount of heat accumulation of each of the obstacle and the normal tissue. In another example, the ultrasonic irradiation intensity of the ultrasonic beam may be controlled according to the temperature and heat accumulation of other tissues in the target object.

In operation 927, when there are a plurality of locations where focusing is to be performed for one lesion, or when there are a plurality of lesions, whether all lesion tissue at the locations where the focusing is to be performed for one lesion and all lesions have been destroyed is determined, the method ends if all lesion tissues have been destroyed, and if all lesion tissues have not been destroyed, the method proceeds to operation 932.

In operation 932, the focus of the ultrasonic irradiation is moved to a portion of a lesion that has not yet been destroyed or to a lesion that has not yet been destroyed, and the method returns to operation 921. Then, in operation 921, the location of the transducer is set to focus the ultrasonic beam at the focus moved to in operation 932. Operations 921 through 932 are repeated until all lesion tissues have been destroyed, and the method ends when all lesion tissues have been destroyed.

According to the examples described above, the most appropriate ultrasonic irradiation plan for treating a patient may be automatically extracted from the medical image data obtained from the medical imaging instrument 100, such as a MRI, a CT, or an ultrasonic imaging instrument, and may be provided to a practitioner. Conventionally, an environment that may be simulated on a computer according to the ultrasonic irradiation plan selected by a practitioner was provided and the result thereof was predictable. However, the plan selected by the practitioner may not be the most appropriate ultrasonic irradiation plan for a patient. According to the examples described above, the most appropriate ultrasonic irradiation plan for treating a patient is made by using various transducer controlling methods such as a location of the transducer, an irradiation intensity, an irradiation time, or a channel combination, and automatically simulating processes of ultrasonic irradiation of a 3D target object model generated on the computer. Accordingly, time spent by a practitioner to make the ultrasonic irradiation plan is greatly reduced.

The examples described above have the following additional features when compared to a method in which a practitioner directly makes an ultrasonic irradiation plan and irradiates the target object accordingly. First, various ultrasonic irradiation plans may be simulated since an infinite number of simulations is theoretically possible, and by selecting the most appropriate ultrasonic irradiation plan for a patient from among so many simulations, the optimum ultrasonic irradiation plan may be made. Second, the ultrasonic irradiation plan made according to the conventional method in which the practitioner directly makes the ultrasonic irradiation plan without such a simulation environment as described above and in which the ultrasonic irradiation plan was made according to the processing situation while directly irradiating the ultrasonic beam to the target object results in a possibility of physical risk to the patient due to trial and error. However, since irradiation simulation automatically proceeds in the virtual environment disclosed in this application, such a risk may be predicted beforehand, and a riskless or less risky irradiation plan is provided, the method is very safe for the patient, and a risk to the practitioner is reduced. Third, when the optimum ultrasonic irradiation plan is selected, various elements and treating times may be considered, and thus an entire treatment time may be reduced, and a risk to the patient may be reduced. Fourth, in a case of moving organs having movement patterns or the organs being located among surrounding obstacles, the conventional ultrasonic irradiation was difficult and dangerous, and thus the treatment would not be able to actively proceed. However, as the simulation environment disclosed in this application considers all cases, ultrasonic irradiation may be performed on many different organs.

As described above, according to the one or more of the above examples, a safe and rapid ultrasonic irradiation operation may be performed on the target object under various conditions by making an optimum ultrasonic irradiation plan based on anatomical features and movements of an organ by using a computer simulation, by providing the plan to a practitioner, and by the practitioner proceeding with ultrasonic irradiation based on the plan.

The ultrasonic irradiation plan making apparatus 200, the receiving unit 210, the information generating unit 220, the image analyzing unit 221, the target object model generating unit 222, the irradiation planning unit 230, the transducer controlling unit 231, the virtual irradiation performing unit 232, the irradiation plan determining unit 233, the ultrasonic irradiation performing apparatus 300, the irradiation determining unit 310, the second receiving unit 311, the comparing unit 312, the irradiating unit 320, the controlling unit 321, and the monitoring unit 322 described above may be implemented using one or more hardware components, one or more software components, or a combination of one or more hardware components and one or more software components.

A hardware component may be, for example, a physical device that physically performs one or more operations, but is not limited thereto. Examples of hardware components include low-pass filters, high-pass filters, band-pass filters, analog-to-digital converters, digital-to-analog converters, and processing devices.

A software component may be implemented, for example, by a processing device controlled by software or instructions to perform one or more operations, but is not limited thereto. A computer, controller, or other control device may cause the processing device to run the software or execute the instructions. One software component may be implemented by one processing device, or two or more software components may be implemented by one processing device, or one software component may be implemented by two or more processing devices, or two or more software components may be implemented by two or more processing devices.

A processing device may be implemented using one or more general-purpose or special-purpose computers, such as, for example, a processor, a controller and an arithmetic logic unit, a digital signal processor, a microcomputer, a field-programmable array, a programmable logic unit, a microprocessor, or any other device capable of running software or executing instructions. The processing device may run an operating system (OS), and may run one or more software applications that operate under the OS. The processing device may access, store, manipulate, process, and create data when running the software or executing the instructions. For simplicity, the singular term “processing device” may be used in the description, but one of ordinary skill in the art will appreciate that a processing device may include multiple processing elements and multiple types of processing elements. For example, a processing device may include one or more processors, or one or more processors and one or more controllers. In addition, different processing configurations are possible, such as parallel processors or multi-core processors.

A processing device configured to implement a software component to perform an operation A may include a processor programmed to run software or execute instructions to control the processor to perform operation A. In addition, a processing device configured to implement a software component to perform an operation A, an operation B, and an operation C may have various configurations, such as, for example, a processor configured to implement a software component to perform operations A, B, and C; a first processor configured to implement a software component to perform operation A, and a second processor configured to implement a software component to perform operations B and C; a first processor configured to implement a software component to perform operations A and B, and a second processor configured to implement a software component to perform operation C; a first processor configured to implement a software component to perform operation A, a second processor configured to implement a software component to perform operation B, and a third processor configured to implement a software component to perform operation C; a first processor configured to implement a software component to perform operations A, B, and C, and a second processor configured to implement a software component to perform operations A, B, and C, or any other configuration of one or more processors each implementing one or more of operations A, B, and C. Although these examples refer to three operations A, B, C, the number of operations that may implemented is not limited to three, but may be any number of operations required to achieve a desired result or perform a desired task.

Software or instructions for controlling a processing device to implement a software component may include a computer program, a piece of code, an instruction, or some combination thereof, for independently or collectively instructing or configuring the processing device to perform one or more desired operations. The software or instructions may include machine code that may be directly executed by the processing device, such as machine code produced by a compiler, and/or higher-level code that may be executed by the processing device using an interpreter. The software or instructions and any associated data, data files, and data structures may be embodied permanently or temporarily in any type of machine, component, physical or virtual equipment, computer storage medium or device, or a propagated signal wave capable of providing instructions or data to or being interpreted by the processing device. The software or instructions and any associated data, data files, and data structures also may be distributed over network-coupled computer systems so that the software or instructions and any associated data, data files, and data structures are stored and executed in a distributed fashion.

For example, the software or instructions and any associated data, data files, and data structures may be recorded, stored, or fixed in one or more non-transitory computer-readable storage media. A non-transitory computer-readable storage medium may be any data storage device that is capable of storing the software or instructions and any associated data, data files, and data structures so that they can be read by a computer system or processing device. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, or any other non-transitory computer-readable storage medium known to one of ordinary skill in the art.

Functional programs, codes, and code segments for implementing the examples disclosed herein can be easily constructed by a programmer skilled in the art to which the examples pertain based on the drawings and their corresponding descriptions as provided herein.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

1. A method of making an ultrasonic irradiation plan, the method comprising:
receiving image data representing anatomical features of a target object;
generating information about at least one portion of the target object that is to be irradiated with ultrasound from the image data representing the anatomical features of the target object; and
making an ultrasonic irradiation plan for irradiating the target object with ultrasound by simulating irradiating the target object with ultrasound based on the generated information.

2. The method of claim 1, wherein the generating of the information comprises designating any one or any combination of a first portion of the target object that is to be irradiated with the ultrasound, a second portion of the target object that the ultrasound is to avoid, and a third portion of the target object that has a characteristic of disrupting propagation of the ultrasound.

3. The method of claim 2, wherein the making of the ultrasonic irradiation plan comprises:
determining whether the ultrasound collides with the second portion or the third portion while virtually irradiating the first portion with the ultrasound using a virtual transducer; and
determining a location of the virtual transducer at which irradiating the target object with the ultrasound is allowed based on a result of the determining of whether the ultrasound collides with the second portion or the third portion.

4. The method of claim 3, further comprising moving the virtual transducer to a plurality of locations;
wherein the determining of whether the ultrasound collides with the second portion or the third portion comprises determining whether the ultrasound collides with the second portion or the third portion at each of the locations of the virtual transducer while virtually irradiating the first portion with the ultrasound at each of the locations of the virtual transducer using the virtual transducer; and
the determining of a location of the virtual transducer at which irradiating the target object with the ultrasound is allowed comprises determining a plurality of locations of the virtual transducer at which irradiating the target object with the ultrasound is allowed based on a result of the determining of whether the ultrasound collides with the second portion or the third portion at each of the locations of the virtual transducer.

5. The method of claim 4, wherein the at least one portion of the target object to be irradiated with the ultrasound comprises a plurality of portions of the target object to be irradiated with the ultrasound;
the making of the ultrasonic irradiation plan further comprises making the ultrasonic irradiation plan by simulating irradiating each of the plurality of portions of the target object with ultrasound based on the generated information; and
the moving, the determining of whether the ultrasound collides with the second portion or the third portion, and the determining of a plurality of locations of the virtual transducer at which irradiating the target object with the ultrasound is allowed are performed for each of the plurality of portions of the target object.

6. The method of claim 5, wherein the making of the irradiation plan further comprises:
selecting one of the plurality of locations of the virtual transducer at which irradiating the target object with the ultrasound is allowed for each of the plurality of portions of the target object based on any one or any combination of an irradiation intensity of an ultrasonic beam irradiated from the virtual transducer, an irradiation time of the ultrasonic beam, and a cooling time for each channel of a plurality of channels of the virtual transducer; and
determining a sequential order in which the plurality of portions of the target object are to be irradiated with the ultrasound.

7. The method of claim 3, wherein the determining of whether the ultrasound collides with the second portion or the third portion comprises determining whether ultrasonic beams radiated to the first portion from all of a plurality of channels of the virtual transducer at the same time collide with the second portion or the third portion.

8. The method of claim 3, wherein the determining of whether the ultrasound collides with the second portion or the third portion comprises determining, for each channel of a plurality of channels of the virtual transducer, whether an ultrasonic beam radiated to the first portion from one channel of the plurality of channels at a time collides with the second portion or the third portion.

9. The method of claim 3, wherein the determining of whether the ultrasound collides with the second portion or the third portion comprises determining, for each channel combination of a plurality of different channel combinations of at least two channels of a plurality of channels of the virtual transducer, whether ultrasonic beams radiated to the first portion from the at least two channels at the same time collide with the second portion or the third portion.

10. The method of claim 3, wherein the generating of the information further comprises designating either one or both of a first critical amount of heat accumulation that will destroy the first portion and a second critical amount of heat accumulation that will destroy the second portion.

11. The method of claim 10, wherein the determining of a location of the virtual transducer at which irradiating the target object with the ultrasound is allowed comprises:
calculating a first amount of heat accumulation in the first portion while virtually irradiating the first portion with the ultrasound using the virtual transducer;
calculating a second amount of heat accumulation in the second portion while virtually irradiating the first portion with the ultrasound using the virtual transducer;
determining whether the first amount of heat accumulation exceeds the first critical amount of heat accumulation;
determining whether the second amount of heat accumulation is less than the second critical amount of heat accumulation; and
determining the location of the virtual transducer at which irradiating the target object with the ultrasound is allowed based on the result of the determining of whether the ultrasound collides with the second portion or the third portion, a result of the determining of whether the first amount of heat accumulation exceeds the first critical amount of heat accumulation, and a result of the determining of whether the second amount of heat accumulation is less than the second critical amount of heat accumulation.

12. The method of claim 1, wherein the generating of the information comprises obtaining a movement pattern and a shape-changing pattern of the at least one portion of the target object from the image data.

13. The method of claim 12, wherein the making of the ultrasonic irradiation plan comprises predicting a location and a shape of the at least one portion of the target object at a point of time at which the target object is to be irradiated with the ultrasound based on the movement pattern and the shape-changing pattern of the at least one portion of the target object.

14. A non-transitory computer-readable storage medium storing a program for controlling a computer to perform the method of claim 1.

15. An apparatus for making an ultrasonic irradiation plan, the apparatus comprising:
a receiving unit configured to receive image data representing anatomical features of a target object;
an information generating unit configured to generate information about at least one portion of the target object that is to be irradiated with ultrasound from the image data representing the anatomical features of the target object; and
a plan making unit configured to make an ultrasonic irradiation plan for irradiating the target object with ultrasound by simulating irradiating the target object with ultrasound based on the generated information.

16. The apparatus of claim 15, wherein the information generating unit is further configured to generate the information by designating any one or any combination of a first portion of the target object that is to be irradiated with the ultrasound, a second portion of the target object that the ultrasound is to avoid, and a third portion of the target object that has a characteristic of disrupting propagation of the ultrasound.

17. An ultrasonic irradiation method comprising:
receiving first image data representing anatomical features of a target object captured at a first point of time, an ultrasonic irradiation plan for irradiating the target object with ultrasound made based on the first image data, and second image data representing anatomical features of the target object captured at a second point of time;
determining whether the ultrasonic irradiation plan made based on the first image data can be used based on a result of comparing the first image data with the second image data; and
irradiating the target object with ultrasound according the ultrasonic irradiation plan made based on the first image data when a result of the determining is that the ultrasonic irradiation plan made based on the first image data can be used.

18. The method of claim 17, further comprising making an ultrasonic irradiation plan for irradiating the target object with ultrasound based on the second image data when the result of the determining is that the ultrasonic irradiation plan made based on the first image data cannot be used.

19. The method of claim 17, further comprising:
modifying the ultrasonic irradiation plan made based on the first image data based on either one or both of third image data representing anatomical features of the target object captured in real time while the target object is being irradiated with the ultrasound, and an amount of heat accumulation due to the irradiating of the ultrasound in at least one portion of the target object where the ultrasound is being irradiated; and
irradiating the target object with ultrasound according to the modified ultrasonic irradiation plan.

20. The method of claim 17, wherein the determining comprises:
comparing a movement pattern and a shape-changing pattern of at least one portion of the target object where the ultrasound is to be irradiated obtained from the first image data with a movement pattern and a shape-changing pattern of the at least one portion of the target object where the ultrasound is to be irradiated obtained from the second image data; and
determining whether the ultrasonic irradiation plan made based on the first image data can be used based on a result of the comparing.
